(19) United States
(12) Patent Application Publication
(10) Pub. No.: US 2026/0284319 A1
(43) Pub. Date: Sep. 24, 2026

(54) SYSTEM AND METHOD OF LIMITING SUBVISIBLE PARTICLES IN A SYRINGE

(71) Applicant: AMGEN INC., Thousand Oaks, CA (US)

(72) Inventors: James Nakamura, Monterey Park, CA (US); Jerome Olivas, Thousand Oaks, CA (US); Ahmed Gouda, Thousand Oaks, CA (US); Austin Dunn, Santa Monica, CA (US); Ryan Oba, Santa Paula, CA (US)

(21) Appl. No.: 19/100,452

(22) PCT Filed: Aug. 29, 2023
(86) PCT No.: PCT/US2023/031351

§ 371 Date: Jan. 31, 2025

Related U.S. Application Data
(60) Provisional application No. 63/402,355, filed on Aug. 30, 2022.

(51) Int. Cl. A61M 5/315; A61M 5/00; A61M 5/31

(52) U.S. Cl. CPC A61M 5/315; A61M 5/002; A61M 2005/3131; A61M 2205/0238; A61M 2207/00

(57) ABSTRACT

A method of limiting subvisible particles in a syringe partially filled with a drug product. The method includes applying a quantity of silicon oil to a syringe barrel of a syringe, and the quantity of silicon oil is in a range of approximately 0.4 mg to approximately 0.6 mg. The syringe barrel includes a proximal end, a distal end, and a reservoir, and a drug product is disposed in a distal end of the reservoir at the distal end of the syringe barrel. The method also includes placing a plunger portion into the syringe barrel to a depth within the syringe barrel and creating an air gap between the plunger portion and the drug product. The plunger portion is spaced from the drug product, and the air gap includes an air gap length in a range of approximately 1.2 mm to approximately 3.5 mm.

### CROSS-REFERENCE TO RELATED APPLICATIONS

This is the United States national phase of International Patent Application No. PCT/US2023/031351, filed Aug. 29, 2023, which This application claims priority to U.S. Provisional Application No. 63/402,355, filed Aug. 30, 2022, each of which is incorporated by reference herein in its entirety.

### FIELD OF DISCLOSURE

The present disclosure relates to a syringe partially filled with a drug product, and more particularly, to a system and method of limiting subvisible particles in a large volume syringe partially filled with a drug product.

### BACKGROUND

Syringes are medical delivery devices used to administer a drug product to a patient. Syringes are often marketed in prefilled form, wherein a set dosage of drug product is already provided therein. In addition, some prefilled syringes are developed specifically for integration with an autojector or other injector device. To integrate with the autojector or other injector device, the prefilled syringes must meet certain requirements. One requirement is the drug product in the prefilled syringe must comply with subvisible particle limits, as defined by the United States Pharmacopeial Convention (USP), USP chapter <787> (USP <787>), Subvisible Particulate Matter in Therapeutic Protein Injections, which include 6,000 particles/container≥10 μm size & 600 particles/container≥25 Nm size. As the biopharmaceutical industry is trending toward higher viscosity and potentially larger volume injections, there is a need for syringes that can deliver larger doses, subcutaneously, while also meeting USP <787> subvisible particle limits per container. Because USP <787> defines these limits per container, rather than per volume, larger syringe systems inherently have a greater challenge adhering to the specified limits. Furthermore, USP <787> does not differentiate between intrinsically generated subvisible particles, such as those generated by silicone oil, in the determination of the total subvisible particle counts.

Silicone oil, as is commonly used in the pharmaceutical industry, is typically applied to an interior surface of a syringe barrel to facilitate plunger stopper movement during injection and/or activation of the device. However, the silicone oil is known to be a primary contributor to subvisible particle counts including subvisible particles (SbVP) in the 10 μm range. In addition, products transported through typical shipping lanes yield higher average subvisible particle counts relative to non-transported controls.

### SUMMARY

In accordance with a first aspect of the present disclosure, a method of limiting subvisible particles in a syringe partially filled with a drug product comprises applying a quantity of silicon oil to a syringe barrel of a syringe, the syringe barrel including a proximal end, a distal end, and a reservoir, wherein a drug product is disposed in a distal end of the reservoir at the distal end of the syringe barrel, and the quantity of silicon oil is in a range of approximately 0.4 mg to approximately 0.6 mg. The method further comprises placing a plunger portion into the syringe barrel to a depth within the syringe barrel, the plunger portion spaced from the drug product; and creating an air gap between the plunger portion and the drug product. The air gap has an air gap length in a range of approximately 1.2 mm to approximately 3.5 mm.

In accordance with a second aspect of the present disclosure, a method of limiting subvisible particles in a syringe partially filled with a drug product comprises applying a quantity of silicon oil to a syringe barrel of a syringe, the syringe barrel including a proximal end, a distal end, and a reservoir, wherein a drug product is disposed in a distal end of the reservoir at the distal end of the syringe barrel, and the quantity of silicon oil is in a range of approximately 0.4 mg to approximately 0.6 mg. The method also includes disposing the syringe into a carton and disposing the carton into a suspension packout system configured to reduce generation of subvisible particles in the syringe during transport. The suspension packout system includes packing material surrounding the carton including the syringe, limiting contact of the carton to the packing material.

In accordance with yet another aspect of the present disclosure, a syringe partially filled with a drug product comprises a syringe barrel including a proximal end, a distal end, and a reservoir, wherein a drug product is disposed in a distal end of the reservoir at the distal end of the syringe barrel. The syringe barrel includes a quantity of silicon oil in a range of approximately 0.4 mg to approximately 0.6 mg, and the quantity of silicon oil is configured to limit an amount of subvisible particles generated in the syringe barrel. The syringe further comprises a plunger portion disposed at a depth within the syringe barrel and spaced from the drug product, and an air gap disposed between the plunger portion and the drug product. The air gap includes an air gap length in a range of approximately 1.2 mm to approximately 3.5 mm.

In further accordance with any one or more of the foregoing aspects, a syringe partially filled with a drug product and a method of limiting subvisible particles in a syringe partially filled with a drug product may include any one or more of the following forms.

In one form, applying a quantity of silicon oil to a syringe barrel may comprise applying the quantity of silicon oil to the syringe barrel with the reservoir having a fill volume in a range of approximately 1.0 mL to approximately 3.0 mL.

In another form, applying a quantity of silicon oil to a syringe barrel may comprise applying the quantity of silicon oil to an interior surface of the syringe barrel along a length of the syringe barrel, facilitating movement of the plunger during an injection of the drug product of the syringe and reducing an amount of subvisible particles generated in the syringe.

In another form, placing a plunger portion into the syringe barrel may comprise placing a stopper into the syringe barrel to a depth within the syringe barrel, the stopper spaced from the drug product.

In one form, creating an air gap between the plunger portion and the drug product may comprise creating the air gap between a most distal point of the plunger portion and a meniscus of the drug product disposed in the distal end of the reservoir.

In another form, the method may further comprise disposing the syringe into a carton and disposing the carton in a suspension packout system configured to reduce generation of subvisible particles in the syringe during transportation of the carton. In one form, disposing the carton in a suspension packout system may comprise disposing the carton within a housing of the suspension packout system and positioning packing material within the housing at least partially around the carton including the syringe, limiting contact of the carton to the packing material during movement. The packing material may include one or more of dunnage, foam, bubble wrap, or cushioning plastic material.

In another form, the method may further comprise placing a plunger portion into the syringe barrel to a depth within the syringe barrel, the plunger portion spaced from the drug product. In addition, placing a plunger portion into the syringe barrel may comprise placing a stopper into the syringe barrel to a depth within the syringe barrel, the stopper spaced from the drug product.

In yet another form, the method may comprise creating an air gap between the plunger portion disposed within the syringe barrel and the drug product, the air gap having an air gap length in a range of approximately 1.2 mm to approximately 3.50 mm.

In still another form, the syringe barrel may further comprise an interior surface and a length, and the quantity of silicon oil may be disposed on the interior surface along at least a portion of the length of the syringe barrel.

In another form, a fill volume of the reservoir of the syringe barrel may be in a range of approximately 1.0 mL to approximately 3.0 mL.

In another form, the plunger portion may be a stopper.

In yet another form, the air gap length may include a distance between a most distal point of the plunger portion and a meniscus of the drug product disposed in the distal end of the reservoir.

In still another form, a suspension packout system may comprise a housing having an interior wall forming an interior area; packing material disposed within the housing adjacent to and/or at least partially around the interior wall; and a carton including the syringe. The carton may be disposed within the packing material and may be limited to contacting the packing material, reducing an amount of subvisible particles generated in the syringe during transport.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omission of elements in some drawings are not necessarily indicative of the presence or absence of particular elements in any of the example embodiments, except as may be explicitly delineated in the corresponding written description. Also, none of the drawings is necessarily to scale.

FIG. 1 is a partially sectional view of a syringe partially filled with a drug product of the present disclosure;

FIG. 2 is graph depicting air gap impact on subvisible particle generation in the syringe of FIG. 1;

FIG. 3 is a graph depicting subvisible particles as a function of air gap length in the syringe of the present disclosure;

FIG. 4 is a graphical representation of silicon oil thickness along a length of a syringe barrel as a function of a distance from a flange of the syringe;

FIG. 5 is graph depicting silicon oil quantity impact on subvisible particle generation;

FIG. 6 is a graph depicting silicon oil nozzle impact on subvisible particle generation in the syringe;

FIG. 7 is a graph depicting fill volume impact on subvisible particle generation in the syringe; and

FIG. 8 is a suspension packout system with the syringe of the present disclosure disposed therein.

### DETAILED DESCRIPTION

Generally, a system and methods of optimizing large volume prefilled syringe production to meet subvisible particle limits defined by USP <787> are disclosed. Production attributes found to most impact and limit the average subvisible particles counts include: (1) silicone quantity disposed in a syringe barrel of a large volume syringe; (2) a packout configuration system for limiting subvisible particles during transport of the syringe; (3) an air gap length disposed between a plunger portion and a drug product disposed in the syringe barrel; and (4) a fill volume of a reservoir of the syringe barrel. More specifically, methods of limiting subvisible particles in a syringe partially filled with a drug product may include one or more of the following steps: (1) applying a quantity of silicon oil to an inside surface of a syringe barrel of a syringe with the quantity of silicon oil in a range of approximately 0.4 mg to 0.6 mg; (2) utilizing the syringe barrel having a fill volume in a range of approximately 1.0 mL to approximately 3.0 mL; (3) placing a plunger portion into the syringe barrel and creating an air gap between the plunger portion and the drug product, the air gap having an air gap length in a range of approximately 1.2 mm to approximately 3.5 mm; and (4) disposing the syringe into a carton and disposing the carton within packing material in a housing of a suspension packout system, reducing generation of subvisible particles in the syringe during movement and/or transport of the carton.

More specifically, and referring now to FIG. 1, a syringe 10 partially filled with a drug product meeting subvisible particle limits is depicted. The syringe 10 includes a syringe barrel 12 including a proximal end 12a, a distal end 12b, an interior surface 12c, a flange 13 disposed near the proximal end 12a, and a reservoir 14. The reservoir 14 may have a larger fill volume, such as a fill volume in a range of approximately 1.0 mL to 3.0 mL, while still meeting the required subvisible particle limits, as explained more below. A drug product 16 is disposed in a distal end 14b of the reservoir 14 at the distal end 12b of the syringe barrel 12. In addition, the interior surface 12c of the syringe barrel 12 has a quantity of silicon oil 18 applied thereto, and the quantity of silicon oil 18 disposed on the interior surface 12c is in a range of approximately 0.4 mg to approximately 0.6 mg. In one example, the optimal quantity of silicon oil 18 disposed on the interior surface 12c is 0.5 mg, as also explained more below. The quantity of silicon oil is configured to limit an amount of subvisible particles generated in the syringe barrel 12.

Still referring to FIG. 1, a plunger portion 20 is disposed at a depth D within the syringe barrel 12, and the plunger portion 20 is spaced from the drug product 16. In one example, the plunger portion 20 is a stopper. Generally, and as will be understood, two stoppering techniques for plunger placement into prefilled syringes may be used to dispose the plunger portion 20 within the syringe barrel 12: (1) vented placement; and (2) vacuum assist. For vented placement, the stopper 20 is placed atop a thin tube (not shown), commonly referred to as a vent tube, that is then inserted into the syringe 10. A plunger placement pin (not shown) advances the stopper 20 down the tube at a depth determined by the pin's length. The vent tube is then retracted, allowing the stopper 20 to expand and fill the syringe diameter. For vacuum assist, a vacuum chamber (not shown) is used to reduce the pressure in the filled syringe 10. Once the target vacuum pressure is reached, the plunger portion 20 is placed inside the syringe barrel 12 to seal the syringe 10 and the chamber is then depressurized. The plunger portion 20 then advances into the syringe barrel 12 to a depth D where the “sealed pressure” equates to ambient, thus determining the plunger portion depth. There is no distinct difference in an average subvisible particle count generated in the syringe 10 using either vacuum assist or the vent tube (vented placement) techniques for disposing the plunger portion 20 in the syringe barrel 12.

The syringe 10 further includes an air gap 22 disposed between the plunger portion 20 and the drug product 18, and the air gap 22 has a length 23 in a range of approximately 1.2 mm to approximately 3.5 mm. More specifically, in one example, the length of the air gap is 1.4 mm, as depicted in FIG. 1. In another example, the length 23 of the air gap 22 is 2.3 mm, and in another example, the air gap length is 3.5 mm. More generally, the length 23 of the air gap 22 includes a distance between a most distal point 24 of the plunger portion 22 and a meniscus 25 of the drug product 18 disposed in the distal end 12b of the reservoir 12. While FIG. 1 depicts a specific length of the air gap 22, it will be understood that the length 23 of the air gap 22 may be any value at or within the range of approximately 1.2 mm to approximately 3.5 mm and still fall within the scope of the present disclosure. In a traditional vacuum assisted plunger placement system, the air gap length 23 is determined by an amount of vacuum pulled prior to the insertion of the plunger portion 20 in the syringe barrel 12. A stronger vacuum pulled on the syringe 10 yields a smaller air gap 22.

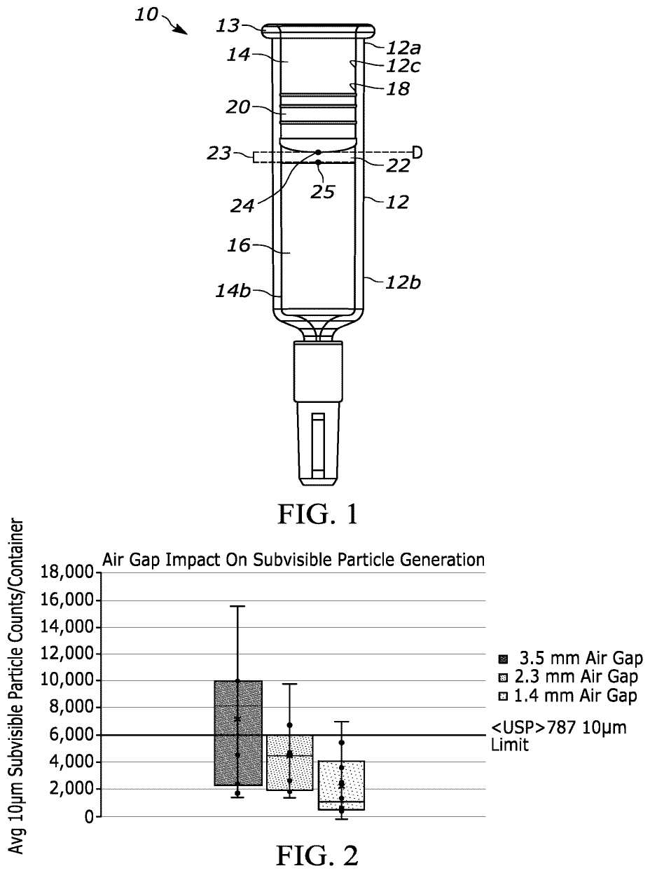

Referring now to FIG. 2, a graph illustrating an impact of the length 23 of the air gap 22 is depicted and, in particular, the proportional relationship between the length 23 of the air gap 22 and subvisible particle generation. Specifically, smaller lengths 23 of air gaps 22 correlate to lower subvisible particle levels in the syringe 10. In addition, data from testing of syringes having each of an air gap length of 1.4 mm, 2.3 mm, and 3.5 mm is provided. For example, subvisible particle counts per container for the syringe 10 having the length of the air gap of 1.4 mm were below the USP limit of 6,000 subvisible particles for the high majority of each sample tested. In addition, subvisible particle counts per container for the syringe 10 having the air gap length of 2.3 mm were also on average below the USP limit of 6,000 subvisible particles, as depicted in FIG. 2. However, when the air gap length is increased to 3.5 mm, the subvisible particle counts per container for the syringe 10 exceed the USP limit of 6,000 subvisible particles for several samples tested, further showing a larger air gap length overall produces more subvisible particles than that of a smaller air gap length.

TABLE 1
Particle Counts Per Container by Air Gap Length and Sample No.

| | 1.4 mm Air Gap | 2.3 mm Air Gap | 3.5 mm Air Gap |
|---|---|---|---|
| | (particles per | (particles per | (particles per |
| Sample ID | container) | container) | container) |
| #1 | 3,750 | 5,530 | 4,580 |
| #2 | 1,020 | 5,860 | 10,020 |
| #3 | 40 | 4,190 | 10,000 |
| #4 | 700 | 9,840 | 2,590 |
| #5 | 550 | 4,950 | 1,480 |
| #6 | 5,560 | 2,750 | 9,640 |
| #7 | 1,510 | 1,490 | 15,580 |
| #8 | 2,640 | 1,960 | 7,030 |
| #9 | 7,130 | 6,780 | 1,800 |
| #10 | 820 | 2,070 | 9,590 |
| Average | 2,388 | 2,612 | 7,231 |
| Standard Deviation | 2,372 | 4,542 | 4,567 |

As demonstrated in Table 1 above, the impact of air gap length to the average particle generation is proportionally related, showing a larger air gap length produces considerably more particles than a smaller air gap length. However, every data set still captures samples that exceeded the USP limit of 6,000, but with less frequency as the air gap length 23 was reduced.

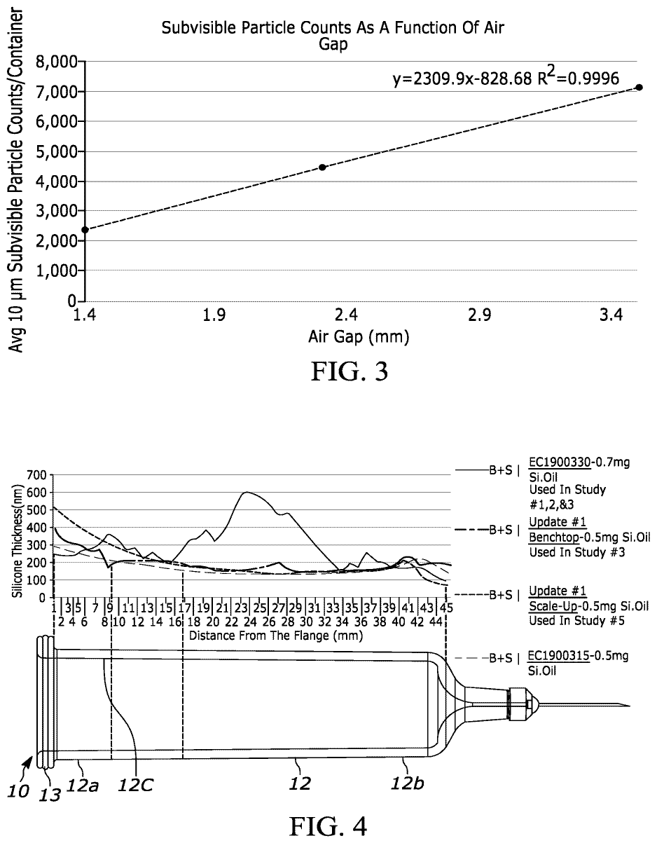

Referring now to FIG. 3, a graph illustrating subvisible particle counts as a function of air gap length is depicted. The average particle counts relative to the associated air gap is well-approximated with a linear fit (R²=0.9996). Therefore, subsequent studies assumed the larger air gap length 23 of 3.5 mm to be the worst case for subvisible particle generation. The 3.5 mm air gap syringe systems produced an average of 7,231 particles with a standard deviation of 4,567 while the 1.40 mm produced an average of 2,372 particles with a standard deviation of 2,388. This is a reduction of 4,859 particles on average, showing a particle generation reduction on the order of 67% and a standard deviation reduction of 48% from the 3.5 mm air gap to the 1.4 mm air gap.

Referring now to FIG. 4, a graphical representation of a quantity, such as a thickness, of the silicon oil 18 disposed on the interior surface 12c of the syringe barrel 12 as a function of a distance from the flange 13 of the syringe barrel 12 is depicted. The bold, dark line in the graph depicts silicone profile data for syringes having a quantity of silicon oil of 0.7 mg, and lighter lines depict silicone profile data for syringes having the quantity of silicon oil 18 of 0.5 mg. The silicon profile data, including the thickness of the quantity of silicon oil 18, is noticeably more uniform with a reduction of the quantity of silicon oil 18 applied to the interior surface 12c across the length of the syringe barrel 12, such as at various distances from the flange 13, for example. A reduced quantity of silicon oil 18 applied is shown to yield a reduction of silicone oil particles in the drug product 16 while still providing an acceptable glide force value for the syringe 10.

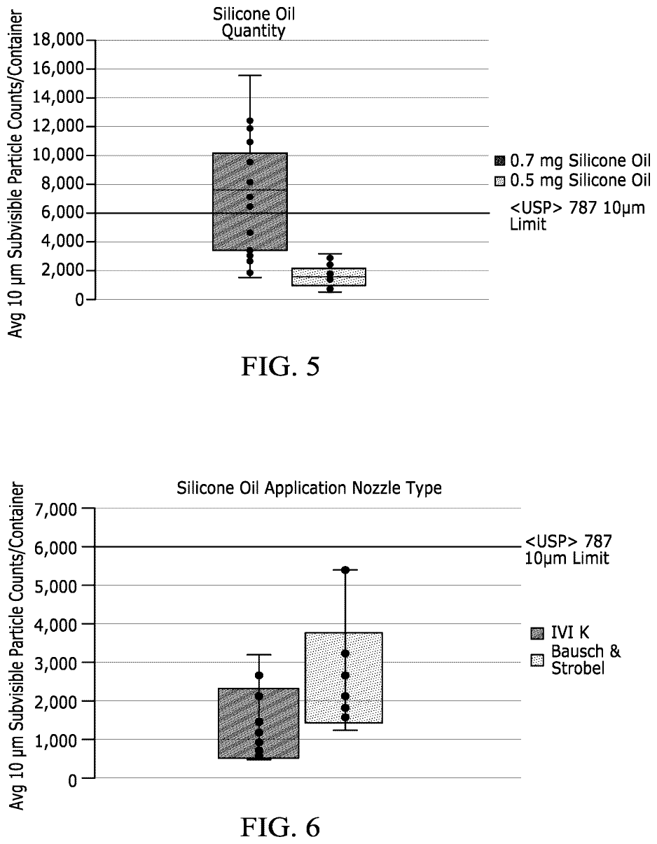

Referring now to FIG. 5, the silicone oil quantity 18 applied to the interior surface 12c of the syringe barrel 12 greatly affects the generation of subvisible particles, such as during transportation of the syringe 10, for example. Generally, the increase in the quantity of silicon oil 18 can aid in the reduction of plunger gliding force values in the syringe 10. Likewise, the increase of the quantity of silicon oil 18 can also lead to an increase in the quantity of silicon oil particles present after transportation of the syringe 10, such as in a PFS post 91.5 hour transportation simulation, preventing the syringe 10 from meeting subvisible particle requirements, for example. As illustrated in FIG. 5, when the syringe included the quantity of silicon oil 18 of 0.7 mg, an average of 7,315 particles with a standard deviation of 4,096 was produced by the syringe, an amount above the required subvisible particle limit of 6,000 particle counts per container, for example. However, when the syringe 10 included the quantity of silicon oil 18 of 0.5 mg, an average of 1,580 particles with a standard deviation of 696 were produced by the syringe 10, which is well below the required subvisible particle limit. When reducing the quantity of silicon oil 18 from 0.7 mg to 0.5 mg, there is reduction of 5,735 particles on average, showing a particle generation reduction of 78%.

Referring now to FIG. 6, a graph illustrating silicon oil nozzle impact on subvisible particle generation in the syringe 10 of the present disclosure is depicted. In production of the syringe 10, various technologies of silicone oil application can be utilized to achieve a target silicone oil profile and quantity with varying tolerances. In one example, a production line used for the syringe 10 having the reservoir 14 with the fill volume of 3.0 mL is capable of utilizing two nozzle systems for applying the quantity of silicon oil 18 to the interior surface 12c of the syringe barrel 12. One nozzle system, e.g., IVEK having a production scale of either an industrial line or a bench top, is a continuous airflow diving nozzle system, and another nozzle system is an intermittent airflow diving nozzle, such as by Bausch+Strobel having a production scale of the industrial line. Samples tested included the quantity of silicone oil 18 of 0.5 mg. As depicted in FIG. 6, the continuous airflow diving nozzle system, e.g., the IVEK nozzle, showed an improvement over the intermittent airflow diving nozzle, e.g., the Bausch+Strobel system, relative to subvisible particle generation. However, both nozzle systems applying the quantity of silicon oil of 0.5 mg to the interior surface 12c of the syringe barrel 12 demonstrated the ability to stay below the USP <787> limits for the 10 μm particle size, e.g., below the limit of 6,000 particles per container, as illustrated in the results of FIG. 6, for example.

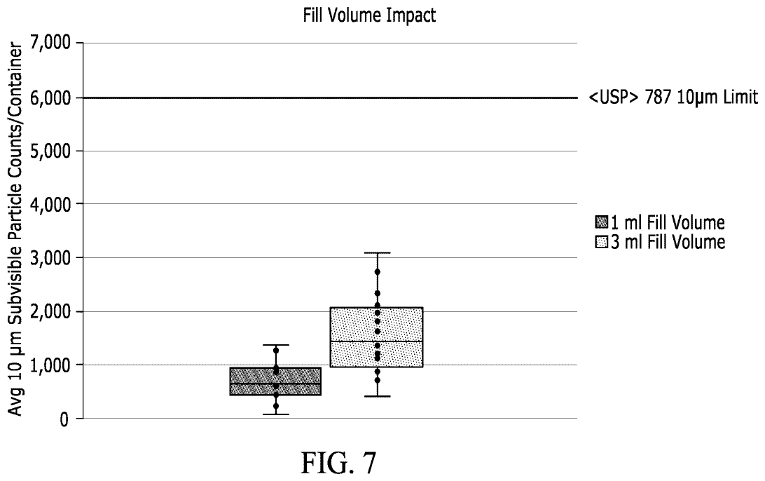

As explained above relative to the syringe 10 of FIG. 1, the reservoir 14 of the syringe barrel 12 may have a larger fill volume, such as a fill volume in a range of approximately 1.0 mL to 3.0 mL, while still meeting the required subvisible particle limits. For example, and referring to FIG. 7, generally a lower fill volume exhibits lower generation of silicone particles. Thus, the fill volume of 3.0 mL for the syringe 10 produces the highest amount of subvisible particle generation, such as post transportation of the syringe 10, for example, compared to lower fill volume of 1.0 mL, which produces the lowest amount of subvisible particle generation. More specifically, and as illustrated in FIG. 7, the syringe 10 having the 3.0 mL fill volume produced an average of 1,580 particles with a standard deviation of 696 while the syringe 10 having the 1.0 mL fill volume produced an average of 703 particles with a standard deviation of 339. This is a reduction of 876 particles on average, showing a particle generation reduction of 55% and a standard deviation reduction of 51% from the 3.0 mL fill volume to the 1.0 mL fill volume, for example.

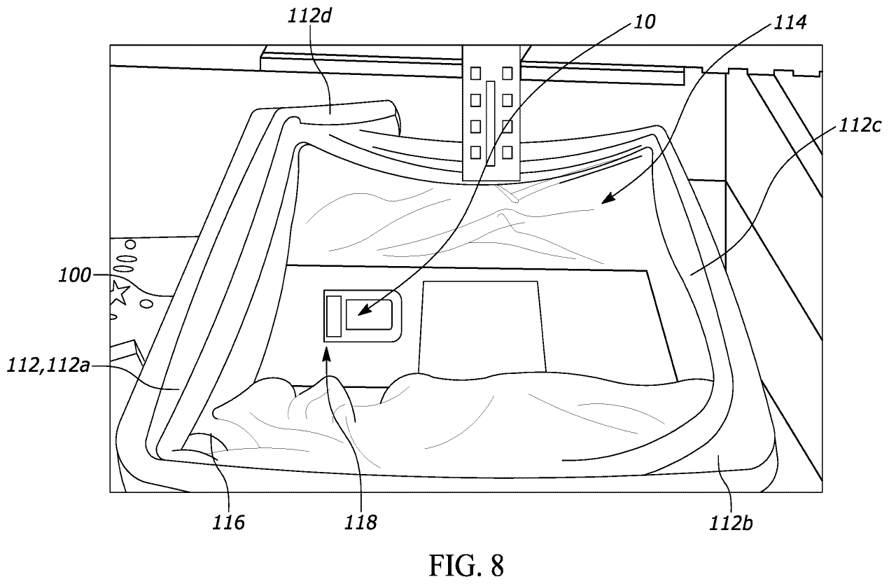

Referring now to FIG. 8, a suspension packout system 100 of the present disclosure is depicted. The suspension packout system 100 includes a housing 110 having an interior wall 112 forming an interior area 114. The suspension packout system 100 further includes packing material disposed within the housing 110 adjacent to and/or at least partially around the interior wall 112. In this example, the packing material 116 is disposed around essentially the entire interior wall 112 of the housing 110. Further, and in this example, there are four interior walls 112a, 112b, 112c, 112d forming the housing 110 that is rectangular in shape. However, it will be appreciated that the housing 110 and thus the interior wall 112 may take the form of various other shapes, such as circular and/or cylindrical and still fall within the scope of the present disclosure. If the housing 110 is circular in the shape, for example, the interior wall 112 is likewise cylindrical in shape, and the packing material 116 is disposed around essentially the entire circumference of the interior wall 112. As will be understood, the packing material 116 may include one or more of dunnage, foam, bubble wrap, peanuts, and/or cushioning plastics material, for example, and still fall within the scope of the present disclosure.

As further depicted in FIG. 8, the suspension packout system 100 also includes and/or is configured to receive a carton 118 including the syringe 10, and typically a plurality of syringes 10 are disposed within the carton 118. So configured, the carton 118 is limited to contacting the packing material 116 only, such as during transportation and/or movement of the carton 118, reducing an amount of subvisible particles generated in the syringe 10 during movement. During testing, the standard, conventional packout system produced an average of 1,580 particles/container with a standard deviation of 696 while the suspension packout system 100 of FIG. 8 produced an average of 566 particles/container with a standard deviation of 405. This is a reduction of 1,015 particles/container on average, showing a particle generation reduction of 64% and a standard deviation reduction of 42% from the standard conventional packout system, for example.

At least in view of the foregoing, various methods of limiting subvisible particles in the syringe 10 and system 100 will be appreciated. For example, and in one example, an exemplary method of limiting subvisible particles in the syringe 10 partially filled with the drug product 16 comprises applying the quantity of silicon oil 18 to the syringe barrel 12 of the syringe 10, such as along the length of the syringe barrel 10, the syringe barrel 10 including the proximal end 12a, the distal end 12b, and the reservoir 14, as explained above. The drug product 16 is disposed in the distal end 14b of the reservoir 14 at the distal end 12b of the syringe barrel 12, and the quantity of silicon oil is in a range of approximately 0.4 mg to approximately 0.6 mg, reducing an amount of subvisible particles generated based on the quantity of silicon oil 18 applied. In one example, applying the quantity of silicon oil 18 comprises applying the quantity of silicon oil 18 to the syringe barrel 12 with the reservoir 14 having a fill volume in a range of approximately 1.0 mL to approximately 3.0 mL. In another example, applying the quantity of silicon oil 18 to the syringe barrel 12 comprises applying the quantity of silicon oil 18 to the interior surface 12c of the syringe barrel 12 and along the length of the syringe barrel, the quantity of silicon oil 18 facilitating movement of the plunger portion 20 during an injection of the drug product 16 in the syringe 10 while simultaneously reducing an amount of subvisible particles generated in the syringe 10.

The method further includes disposing the carton 118 including the syringe 10 into the suspension packout system 100 configured to reduce generation of subvisible particles in the syringe 10 during transportation. The suspension packout system 100 includes the packing material 116 at least partially spaced around the carton 118 including the syringe 10, limiting contact of the carton 118 including the syringe 10 to the packing material 116. In one example, limiting contact of the carton 118 including the syringe 10 to packing material 116 comprises limiting the contact of the carton 118 to one or more of dunnage, foam, bubble wrap, or cushioning plastic material, maintaining a position of the carton 118 within the suspension packout system 100 during movement of the suspension packout system 100 and/or carton 118.

The method may also include placing the plunger portion 20 into the syringe barrel 12 to the depth D within the syringe barrel 12, such that the plunger portion 20 is spaced from the drug product 16. In some examples, placing the plunger portion 20 into the syringe barrel 12 comprises placing a stopper 20 into the syringe barrel 12 to the depth D within the syringe barrel 12, the stopper 20 spaced from the drug product 16. The method may still also include creating the air gap 22 between the plunger portion 20 disposed within the syringe barrel 12 and the drug product 16, and the air gap includes an air gap length 23 in a range of approximately 1.2 mm to approximately 3.5 mm, with the air gap length 23 being 1.4 mm in one example. In another example, the air gap length 23 is 2.3 mm, and in another example, the air gap length 23 is 3.5 mm.

Another exemplary method of limiting subvisible particles in the syringe 10 partially filled with the drug product 16 comprises applying the quantity of silicon oil 18 to the syringe barrel 12 of the syringe 10, the syringe barrel 12 including the proximal end 12a, the distal end 12b, and the reservoir 14. The drug product 16 is disposed in the distal end 14b of the reservoir 14 at the distal end 12b of the syringe barrel 12, and the quantity of silicon oil 17 is in a range of approximately 0.4 mg to approximately 0.6 mg. The method also includes placing the plunger portion 20 into the syringe barrel 12 to the depth D within the syringe barrel 12 and creating the air gap 22 between the plunger portion 20 and the drug product 16, the air gap 22 having an air gap length 23 in a range of approximately 1.2 mm to approximately 3.5 mm.

In some examples for this method, applying the quantity of silicon oil 18 to the syringe barrel 12 again comprises applying the quantity of silicon oil 18 to the syringe barrel 12 with the reservoir 14 having a fill volume in a range of approximately 1.0 mL to approximately 3.0 mL. In one example, the fill volume is 3.0 mL, as explained above, for example. In addition, applying the quantity of silicon oil 18 to the syringe barrel 12 may further include applying the quantity of silicon oil 18 to the interior surface 12c of the syringe barrel 12, facilitating movement of the plunger portion 20 during an injection of the drug product of the syringe and reducing an amount of subvisible particles generated in the syringe 10.

Further, placing the plunger portion 20 into the syringe barrel comprises placing the stopper 20 into the syringe barrel 12 to the depth D within the syringe barrel 12, the stopper 20 spaced from the drug product 16. In addition, creating the air gap 22 between the plunger portion 20 and the drug product 16 may include creating the air gap 22 between the most distal point of the plunger portion 20 and the meniscus 25 of the drug product 16 disposed in the distal end 12b of the reservoir 12. Still further, after creating the air gap 22 between the plunger portion 20 and the drug product 16, the method may further include disposing the syringe 10 into the carton 118 and then disposing the carton 118 in the suspension packout system 100 configured to reduce generation of subvisible particles in the syringe 10 during movement of the carton 118. Moreover, disposing the carton 118 in the suspension packout system 100 may include disposing the carton 118 within the housing 110 of the suspension packout system 100 and positioning packing material 116 within the housing 110 at least partially around the carton 118 including the syringe 10, limiting contact of the carton 118 to the packing material 116 during movement. The packing material 116 may include one or more of dunnage, foam, or cushioning plastic material.

Various experiments related to the syringe 10, the system 100, and methods of the present disclosure described above were conducted, confirming the various results and advantages of the present disclosure. More specifically, in executing the experiments, exemplary sample syringes used included an OMPI 3.0 mL syringe and a Daikyo-Seiko 3.0 mL Flurotec coated Plungee. Sample syringes were filled with 3.1 mL drug product and finished using the automated stopper placement technique described above, for example. The sample syringes were then packaged as pre-filled syringes into packaging, consisting of three (3) fourteen (14) count rondo trays packaged into the standard Carton for syringe systems. The samples syringes were then subjected to a transportation simulation, and then manually extruded and measured for subvisible particle counts per MET-404639 (HIAC) and MET-403619 (MFI). Additionally, other methodologies of SbVP quantification were utilized such as Microscopic Particle Count Test for Quantifying Liquid Borne Subvisible Particle Matter (MET-401787) and HORIZON Aura System from Halo Labs. This series of studies utilized the transportation test sequence typically executed as part of transport operational qualification (TOQ) studies. This includes a “91.5 hour” simulation transportation test, in which syringes are subjected to many, repeated drops, significant vibration, and pressure changes mimicking the transportation factors that will occur during actual transportation of the syringes. Notable increases in subvisible particles in the syringes exceeding acceptable limits, for example, occur after such agitation. For this study, a cycling chamber was programmed to maintain a constant 1800 for the duration of the simulation, as this is intended to simulate the transport of the samples prior to subvisible particle testing and not intended to test the insulated shippers' ability to maintain 2-800. A summary of data from executed experiments is provided below in Table 2 below.

TABLE 2
Summary of Executed Experiments for Sample Syringe

| | | | Average | SD | MAX | CONTROL | |
|---|---|---|---|---|---|---|---|
| Study | Month of | | Particles/ | Particles/ | Particles/ | Particles/ | # Failing |
| # | Testing | Sample Description | Container | Container | Container | Container | USP 787 |
| Study | 2020 July | 0.7 mg, Industrial Line, 3.5 | 9733 | 2605 | 13770 | 245 | 14 out of 15 |
| #1 | | mm air gap | | | | | |
| Study | 2020 August | 0.7 mg, Industrial Line, 3.5 | 7399 | 3814 | 12470 | 275 | 6 out of 10 |
| #2 | | mm air gap | | | | | |
| | 2020 August | 0.7 mg, Industrial Line, 3.5 | 7231 | 4567 | 15580 | 401 | 6 out of 10 |
| | | mm air gap | | | | | |
| | 2020 August | 0.7 mg, Industrial Line, 2.3 | 4542 | 2612 | 9840 | — | 2 out of 10 |
| | | mm air gap | | | | | |
| | 2020 August | 0.7 mg, Industrial Line, 1.4 | 2372 | 2388 | 7130 | — | 1 out of 10 |
| | | mm air gap | | | | | |
| Study | 2020 October | 0.7 mg, Industrial Line, 3.5 | 9263 | 7367 | 24000 | 202 | 4 out of 10 |
| #3 | | mm air gap | | | | | |
| | 2020 October | 0.5 mg, Bench Top, 3.5 mm air | 1729 | 1084 | 3640 | 177 | 0 out of 10 |
| | | gap | | | | | |
| Study | 2020 November | 0.5 mg, Bench Top, 3.5 mm air | 2991 | 2665 | 11420 | — | 1 out of 15 |
| #4 | | gap | | | | | |
| | 2020 November | 0.5 mg, Industrial Line, B + S^{a}, | 4725 | 3626 | 15456 | — | 3 out of 15 |
| | | 3.5 mm air gap | | | | | |
| Study | 2020 December | 0.5 mg, Industrial Line, B + S^{a}, | 2109 | 927 | 3920 | 182 | 0 out of 15 |
| #5 | | 3.5 mm air gap, Day 1 | | | | | |
| | 2020 December | 0.5 mg, Industrial Line, B + S^{a}, | 2662 | 1580 | 5430 | — | 0 out of 10 |
| | | 3.5 mm air gap, Day 3 | | | | | |
| Study | 2021 February | 0.5 mg, Industrial Line, B + S^{a}, | 1797 | 940 | 3460 | 235 | 0 out of 20 |
| #6 | | 3.5 mm air gap, 3 mL fill, | | | | | |
| | | Standard Pack, Day 1 | | | | | |
| | 2021 February | 0.5 mg, Industrial Line, B + S^{a} | 1580 | 696 | 3130 | — | 0 out of 20 |
| | | 3.5 mm air gap, 3 mL fill, | | | | | |
| | | Standard Pack, Day 3 | | | | | |
| | 2021 February | 0.5 mg, Industrial Line, B + S^{a}, | 903 | 505 | 2200 | — | 0 out of 20 |
| | | 3.5 mm air gap, 3 mL fill, | | | | | |
| | | Suspension Pack, Day 1 | | | | | |
| | 2021 February | 0.5 mg, Industrial Line, B + S^{a}, | 565 | 405 | 1860 | — | 0 out of 20 |
| | | 3.5 mm air gap, 3 mL fill, | | | | | |
| | | Suspension Pack, Day 3 | | | | | |
| | 2021 February | 0.5 mg, Industrial Line, B + S^{a}, | 777 | 360 | 1620 | — | 0 out of 40 |
| | | 3.5 mm air gap, 1 mL fill, | | | | | |
| | | Standard Pack, Day 1 + Day 3 | | | | | |

As demonstrated in Table 2, the sample syringes having a fill volume of 3 mL, the air gap length of 3.5 mm, and the quantity of silicon oil of 0.5 mg had no sample failing the subvisible particle limit requirement, for example.

Further, while various attributes potentially related to reducing particle counts to mitigate and/or limit subvisible particles generated in the syringes were investigated, the following attributes demonstrated the greatest reduction of particle counts: (1) silicone quantity; (2) packout configuration; (3) air gap length; and (4) fill volume. Table 3 below lists the attributes having the greatest impact and, thus, used in the system and methods of the present disclosure, for example. The attributes found to not have any impact to particle generation are also included in the table.

TABLE 3
Impact Magnitude of Experiment Attributes Based on Particle Count Reduction

| Impact | | Reduction to | Reduction to | |
|---|---|---|---|---|
| Magnitude | | Particle | Particle Counts | |
| (Rank) | Attribute | Counts (avg) | (St. Dev) | Comment |
| 1 | Silicone Quantity | 78% | 83% | 0.7 mg to 0.5 mg |
| 2 | Packout | 64% | 42% | Standard to |
| | Configuration | | | Suspension Packout |
| 3 | Air Gap Size | 67% | 48% | 3.5 mm to 1.4 mm |
| 4 | Fill Volume | 55% | 51% | 3.0 mL to 1.2 mL |
| 5 | Transportation Type | — | — | Attribute does not |
| 6 | Silicone Nozzle Type | — | — | produce measurable |
| 7 | Stoppering Type | — | — | impact to particle |
| 8 | Day 1 v. Day 3 | — | — | generation |
| | HIAC Testing | | | |
| | Post Trans. Sim. | | | |

At least in view of the foregoing, the following various advantages of the system and methods of the present disclosure will be appreciated. The syringe 10 and related methods apply and maintain an optimal quantity of silicon oil 18 across the desired length of the syringe barrel 12 to maintain lubricity for facilitating movement of the plunger portion 20 (e.g., during an injection) while simultaneously reducing subvisible particles in the syringe 10, meeting required subvisible particle limits. In addition, the packing material 116 disposed around the carton 118 including the syringe 10 of the suspension packout system 100 mitigates and/or lowers impact to the syringe 10 during transport and, in particular, during any drops, which were found to often generate the most subvisible particles, limiting subvisible particles in the syringe 10. In addition, reducing and/or having the air gap length 22 in the range of approximately 1.2 mm and approximately 3.5 mm results in less surface area of the syringe barrel 12 to move liquid within the syringe 10, for example, resulting in a reduction of the amount of siliconized surface area of the syringe barrel 12 that is exposed to the drug product 16. Further, an amount of liquid movement used to potentially remove some of the silicon oil 18 off the interior surface of the syringe barrel 12 is less due to the reduced air gap length 22 and surface area of the syringe barrel 12, resulting in a more uniform quantity of silicon oil 18 being maintained on the interior surface 12c of the syringe barrel 12, for example. Still further, by having the reservoir 14 with the lower fill volume in the range of approximately 1.0 mL to approximately 3.0 mL, the plunger portion 20 is moved to lower depth D, for example, within the syringe barrel 12, again reducing the surface area within the syringe barrel 12 exposed. The amount of subvisible particles thus caused by the quantity of silicon oil 18 is also reduced.

Moreover, by implementing the quantity of silicon oil 18 described above within the syringe barrel 12 of the syringe 10 and disposing the syringe 10 with such silicon oil 18 features in the carton 118, and the carton 118 within the packing material 116 of the suspension packout system 100, a 94% reduction is average subvisible particle generation in the syringe 10 was achieved along with an 84% standard deviation. In addition, subvisible particle counts are consistently below the defined limits (e.g., explained above) for the new features of the present disclosure.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed.

The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-?4B7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNF? monoclonal antibody); Reopro® (abciximab, anti-GP lib/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-?4 integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2R? mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNF? mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-?5?1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFN? mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG? mAb (MDX-1307); anti-mesothelin dsFv-PE38 conjugate (CAT-5001); anti-PD1mAb (MDX-1106 (ONO-4538)); anti-PDGFR? antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF? monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vll (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tightjunction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A method of limiting subvisible particles in a syringe partially filled with a drug product, the method comprising:
applying a quantity of silicon oil to a syringe barrel of a syringe, the syringe barrel including a proximal end, a distal end, and a reservoir, wherein a drug product is disposed in a distal end of the reservoir at the distal end of the syringe barrel, and the quantity of silicon oil is in a range of approximately 0.4 mg to approximately 0.6 mg;
placing a plunger portion into the syringe barrel to a depth within the syringe barrel, the plunger portion spaced from the drug product; and
creating an air gap between the plunger portion and the drug product, the air gap having an air gap length in a range of approximately 1.2 mm to approximately 3.5 mm.

2. The method of claim 2, wherein applying a quantity of silicon oil to a syringe barrel comprises applying the quantity of silicon oil to the syringe barrel with the reservoir having a fill volume in a range of approximately 1.0 mL to approximately 3.0 mL.

3. The method of claim 1, wherein applying a quantity of silicon oil to a syringe barrel comprises applying the quantity of silicon oil to an interior surface of the syringe barrel along a length of the syringe barrel, facilitating movement of the plunger during an injection of the drug product of the syringe and reducing an amount of subvisible particles generated in the syringe.

4. The method of claim 1, wherein placing a plunger portion into the syringe barrel comprises placing a stopper into the syringe barrel to a depth within the syringe barrel, the stopper spaced from the drug product.

5. The method of claim 1, wherein creating an air gap between the plunger portion and the drug product comprises creating the air gap between a most distal point of the plunger portion and a meniscus of the drug product disposed in the distal end of the reservoir.

6. The method of claim 1, wherein after creating an air gap between the plunger portion and the drug product, the method further comprises disposing the syringe into a carton and disposing the carton in a suspension packout system configured to reduce generation of subvisible particles in the syringe during transportation of the carton.

7. The method of claim 6, wherein disposing the carton in a suspension packout system comprises disposing the carton within a housing of the suspension packout system and positioning packing material within the housing at least partially around the carton including the syringe, limiting contact of the carton to the packing material during movement, the packing material including one or more of dunnage, foam, bubble wrap, or cushioning plastic material.

8. A method of limiting subvisible particles in a syringe partially filled with a drug product, the method comprising:
applying a quantity of silicon oil to a syringe barrel of a syringe, the syringe barrel including a proximal end, a distal end, and a reservoir, wherein a drug product is disposed in a distal end of the reservoir at the distal end of the syringe barrel, and the quantity of silicon oil is in a range of approximately 0.4 mg to approximately 0.6 mg;
disposing the syringe into a carton; and
disposing the carton into a suspension packout system configured to reduce generation of subvisible particles in the syringe during transport, the suspension packout system including packing material surrounding the carton including the syringe, limiting contact of the carton to the packing material.

9. The method of claim 9, wherein limiting contact of the carton to packing material comprises limiting the contact of the carton to one or more of dunnage, foam, bubble wrap, or cushioning plastic material, maintaining a position of the carton within the suspension packout system during transport.

10. The method of claim 8, further comprising placing a plunger portion into the syringe barrel to a depth within the syringe barrel, the plunger portion spaced from the drug product.

11. The method of claim 10, wherein placing a plunger portion into the syringe barrel comprises placing a stopper into the syringe barrel to a depth within the syringe barrel, the stopper spaced from the drug product.

12. The method of claim 10, further comprising creating an air gap between the plunger portion disposed within the syringe barrel and the drug product, the air gap having an air gap length in a range of approximately 1.2 mm to approximately 3.5 mm.

13. The method of claim 8, wherein applying a quantity of silicon oil comprises applying the quantity of silicon oil to the syringe barrel with the reservoir having a fill volume in a range of approximately 1.0 mL to approximately 3.0 mL.

14. The method of claim 8, wherein applying a quantity of silicon oil to a syringe barrel comprises applying the quantity of silicon oil to an interior surface of the syringe barrel, the quantity of silicon oil facilitating movement of the plunger during an injection of the drug product in the syringe while reducing an amount of subvisible particles generated in the syringe.

15. A syringe partially filled with a drug product, the syringe comprising:
a syringe barrel including a proximal end, a distal end, and a reservoir, wherein a drug product is disposed in a distal end of the reservoir at the distal end of the syringe barrel, the syringe barrel having a quantity of silicon oil in a range of approximately 0.4 mg to approximately 0.6 mg, the quantity of silicon oil configured to limit an amount of subvisible particles generated in the syringe barrel;
a plunger portion disposed at a depth within the syringe barrel, the plunger portion spaced from the drug product; and
an air gap disposed between the plunger portion and the drug product, the air gap having an air gap length in a range of approximately 1.2 mm to approximately 3.5 mm.

16. The syringe of claim 15, the syringe barrel further comprising an interior surface and a length, and the quantity of silicon oil disposed on the interior surface along the length of the syringe barrel.

17. The syringe of claim 15, wherein a fill volume of the reservoir of the syringe barrel is in a range of approximately 1.0 mL to approximately 3.0 mL.

18. The syringe of claim 15, wherein the plunger portion is a stopper.

19. The syringe of claim 15, wherein the air gap length includes a distance between a most distal point of the plunger portion and a meniscus of the drug product disposed in the distal end of the reservoir.

20. A suspension packout system comprising:
a housing having an interior wall forming an interior area;
packing material disposed within the housing adjacent to and/or at least partially around the interior wall; and
a carton including the syringe of claim 15, the carton disposed within the packing material and limited to contacting the packing material, reducing an amount of subvisible particles generated in the syringe during transport.
